(19) United States
(12) Patent Application Publication
(10) Pub. No.: US 2005/0221363 A1
(43) Pub. Date: Oct. 6, 2005

(54) Universal shotgun assay

(75) Inventors: Michael D. Spain, Austin, TX (US); Mark B. Chandler, Austin, TX (US)

Correspondence Address: KATTEN MUCHIN ROSENMAN LLP, 525 WEST MONROE STREET, CHICAGO, IL, 60661-3693, US

(73) Assignee: Rules-Based Medicine, Inc., Austin, TX (US)

(21) Appl. No.: 11/094,366

(22) Filed: Mar. 31, 2005

Related U.S. Application Data
(60) Provisional application No. 60/558,136, filed on Apr. 1, 2004.

(51) Int. Cl. C12Q 1/68; G01N 33/53

(52) U.S. Cl. 435/6; 435/7.1

(57) ABSTRACT

A method for the multiplexed diagnosis of a plurality of different biomolecules in a fluid sample substantially simultaneously is provided. In accordance with a method of the invention, a substantial fraction of biomolecules in a fluid sample are complexed with a universal label and a secondary labeling reagent. Flow cytometric measurements may be used to identify and quantify, in real-time, by detecting the secondary reagent and universal label present in any of said complexes. The inventive technology enables the simultaneous, and automated, detection and interpretation of multiple biomolecules while also reducing the cost of performing diagnostic and genetic assays.

### PRIORITY

This application claims priority to U.S. provisional patent application Ser. No. 60/558,136, filed Apr. 01, 2004, the entire disclosure of which is incorporated herein by reference.

### FIELD OF THE INVENTION

The invention relates generally to an analytical system and/or method and, more particularly, to a substantially simultaneous and multiplexed, multi-analyte or genetic analysis of clinical specimens.

### BACKGROUND OF THE INVENTION

Analysis of clinical specimens is important in science and medicine. A wide variety of assays to determine qualitative and/or quantitative characteristics of a specimen are known in the art, however, few are able to provide detection of multiple analytes, or separately identifiable characteristics of one or more analytes, through single-step assay processes. To the extent possible, the current “multiplex” assays have not yielded satisfactory results. Some of the reason for these disappointing results may be attributed to the use of an analyte-specific reagent to first tag a target biomolecule and then a second, and different, analyte-specific reagent to report or detect the analyte.

A requirement in the aforementioned case for two specific reagents to the same analyte can add cost and introduce inherent limitations of available reagents. Clearly, therefore, it would be an improvement in the art to have adequate apparatus and methods for reliably performing real-time multiple determinations of a plurality of different analytes, substantially simultaneously, which obviates the need for two specific reagents for a given analyte.

### SUMMARY OF THE INVENTION

In one embodiment of the present invention, a method of revealing the identities of a plurality of different analytes present in a fluid sample is provided, comprising (a) contacting a fluid sample, which is suspected of harboring a plurality of different analytes, with a universal reagent capable of binding to all or substantially all of said plurality of different analytes, under conditions effective to label all or substantially all of said plurality of different analytes with said universal reagent; (b) contacting said plurality of different analytes labeled with said universal reagent with a collection of distinguishable subpopulations of secondary reagent, each secondary reagent of a given distinguishable subpopulation being designed to interact selectively with a given analyte of said plurality of different analytes, under conditions effective to provide a plurality of complexes, each complex comprised of said distinguishable secondary reagent and said given analyte labeled with said universal reagent; and (c) detecting through said universal reagent the existence of a complex and determining through said distinguishable secondary reagent the nature of said given analyte found therein, thereby revealing the identities of the plurality of different analytes present in the fluid sample. It will be understood that quantities of the plurality of different analytes can likewise be determined.

The universal reagent may comprises one or more analyte binding moieties and one or more detectable labels; the detectable label may further comprises a fluorescent group, a radioactive atom, or a combination thereof. The universal reagent may comprise a metal complex and the metal complex may comprise a platinum metal complex labeled with a fluorescent group. Further, the metal complex may comprise a metalloporphyrin or platinum metal complex including (i) biotin or a biotin-containing moiety and (ii) a leaving group. The analyte binding moieties may be a protein, nucleic acid, or a lipid. In some embodiments, the method the collection of distinguishable subpopulations of secondary reagent comprises planar substrates, magnetic beads, or fluorescent microspheres; polypeptides, polynucleotides, or combinations thereof; antibodies or the binding regions thereof. The fluorescent microspheres can form distinguishable subpopulations through distinguishable combinations of two or more fluorophores. Step (c) may optionally be carried out in a flow analyzer and the universal reagent and the secondary reagent each may exhibit one or more characteristic fluorescence emission classification parameters. Alternatively, the universal reagents may differ from the secondary reagents in an intensity of at least one fluorescence emission classification parameter. One of the given analytes may comprise a hormone, antigen, cytokine, antibody or oligopeptide; genomic DNA, cDNA, oligonucleotide, RNA, and RNA nucleotides. Fluid samples of the instant mentod can be selected from the group consisting of plasma, serum, tears, mucus, saliva, urine, pleural fluid, spinal fluid, gastric fluid, sweat, semen, vaginal secretion, broncioalveolar lavage, sputum, breath condensate, peritoneal fluid, joint fluid, fluid from ulcers and other surface eruptions, blisters, abscesses, and extracts of tissues including biopsies of normal, malignant, and suspect tissues. The fluid sample may also comprise a cell lysate; be obtained from a mammal; or from a human. The method may further comprise the step of removing unbound secondary reagent prior to performing step (c). One or more universal or secondary reagents may be coupled to a solid support, which may comprise a microsphere, microchip, biochip, or any planar array.

In another embodiment of the instant invention, a method of revealing the identities of a plurality of different analytes present in a fluid sample is provided comprising (a) contacting a fluid sample, which is suspected of harboring a plurality of different analytes, with a universal reagent capable of binding to all or substantially all of said plurality of different analytes, under conditions effective to label all or substantially all of said plurality of different analytes with said universal reagent; (b) contacting said plurality of different analytes labeled with said universal reagent with a collection of distinguishable subpopulations of secondary reagent, each secondary reagent of a given distinguishable subpopulation being designed to interact selectively with a given analyte of said plurality of different analytes, under conditions effective to provide a plurality of complexes, each complex comprised of said distinguishable secondary reagent and said given analyte labeled with said universal reagent; (c) contacting said plurality of complexes with excess reporter reagent, the reporter reagent having an affinity for at least a portion of said universal reagent and further comprised of a detectable label or cleavable group, under conditions effective to provide a plurality of complexes, each complex bearing a detectable label or cleavable group; and (d) detecting through said detectable label the existence of a complex and determining through said distinguishable secondary reagent the nature of said given analyte found therein, thereby revealing the identities of the plurality of different analytes present in the fluid sample. The universal reagent may comprise a metal complex, and the metal complex may comprise a platinum metal complex including biotin or a biotin-containing moiety. In some embodiments, the reporter reagent is avidin bound to a detectable label. The detectable label can give rise to fluorescence, chemiluminescence, or a color change reaction and a step of removing unbound reporter reagent may be performed prior to step (d).

In yet another embodiment of the present invention, a method of arriving at a liklihood that a subject is suffering from or may later develop one or more diseases is provided, comprising (a) contacting a fluid sample, which is suspected of harboring a plurality of different analytes, with a universal reagent capable of binding to all or substantially all of said plurality of different analytes, under conditions effective to label all or substantially all of said plurality of different analytes with said universal reagent; (b) contacting said plurality of different analytes labeled with said universal reagent with a collection of distinguishable subpopulations of secondary reagent, each secondary reagent of a given distinguishable subpopulation being designed to interact selectively with a given analyte of said plurality of different analytes, under conditions effective to provide a plurality of complexes, each complex comprised of said distinguishable secondary reagent and said given analyte labeled with said universal reagent; (c) contacting said plurality of complexes with excess reporter reagent, the reporter reagent having an affinity for at least a portion of said universal reagent and further comprised of a detectable label or cleavable group, under conditions effective to provide a plurality of complexes, each complex bearing a detectable label or cleavable group; (d) detecting through said detectable label the existence of a complex and determining through said distinguishable secondary reagent the nature of said given analyte found therein, thereby revealing the identities and quantities of the plurality of different analytes present in the fluid sample; (e) comparing analyte data generated from step (d) with a database of accumulated analyte data, which accumulated analyte date comprises a compilation of analyte data obtained from a statistically significant number of subjects suffering from or who later develop one or more diseases; (f) optionally arriving at a likelihood that such subject is suffering from or will later develop one or more diseases based, at least in part, on the results of said comparison.

In still yet another method of generating an animal model of a disease is provided, comprising (a) contacting a fluid sample, which is suspected of harboring a plurality of different analytes, with a universal reagent capable of binding to all or substantially all of said plurality of different analytes, under conditions effective to label all or substantially all of said plurality of different analytes with said universal reagent; (b) contacting said plurality of different analytes labeled with said universal reagent with a collection of distinguishable subpopulations of secondary reagent, each secondary reagent of a given distinguishable subpopulation being designed to interact selectively with a given analyte of said plurality of different analytes, under conditions effective to provide a plurality of complexes, each complex comprised of said distinguishable secondary reagent and said given analyte labeled with said universal reagent; (c) contacting said plurality of complexes with excess reporter reagent, the reporter reagent having an affinity for at least a portion of said universal reagent and further comprised of a detectable label, under conditions effective to provide a plurality of complexes, each complex bearing a detectable label or cleavable group; (d) detecting through said detectable label the existence of a complex and determining through said distinguishable secondary reagent the nature of said given analyte found therein, thereby revealing the identities of the plurality of different analytes present in the fluid sample; (e) determining a relationship between one or more given analytes and the disease of the population of subjects whose accumulated biochemical analyte data share similar features; and (f) genetically manipulating an animal to express the one or more given analytes related to the disease of interest. The animal may be a rodent.

In further still yet another embodiment of the present invention, a method of determining the effects of drud admisstration on a subject epressing one or more given analytes is provided, comprising (a) contacting fluid samples from a subject both before and after administration of a drug, respectively, which samples are suspected of harboring a plurality of different analytes, with a universal reagent capable of binding to all or substantially all of said plurality of different analytes, under conditions effective to label all or substantially all of said plurality of different analytes, under conditions effective to label all or substantially all of said plurality of different analytes with said universal reagent; (b) contacting said plurality of different analytes labeled with said universal reagent with a collection of distinguishable subpopulations of secondary reagent, each secondary reagent of a given distinguishable subpopulation being designed to interact selectively with a given analyte of said plurality of different analytes, under conditions effective to provide a plurality of complexes, each complex comprised of said distinguishable secondary reagent and said given analyte labeled with said universal reagent; (c) contacting said plurality of complexes with excess reporter reagent, the reporter reagent having an affinity for at least a portion of said universal reagent and further comprised of a detectable label, under conditions effective to provide a plurality of complexes, each complex bearing a detectable label or cleavable group; (d) detecting through said detectable label the existence of a complex and determining through said distinguishable secondary reagent the nature of said given analyte found therein, thereby revealing the identities of the plurality of different analytes present in the fluid samples; and (e) comparing the analyte data generated from the sample taken before administration of the drug with the analyte data generated from the sample taken after administration of the drug to provide the effect(s), if any, of said drug administration on the analyte data expressed by the subject.

In further still yet another embodiment of the present invention, a computer implemented method for generating information on a disease is provided, comprising: (a) contacting a fluid sample, which is suspected of harboring a plurality of different analytes, with a universal reagent capable of binding to all or substantially all of said plurality of different analytes, under conditions effective to label all or substantially all of said plurality of different analytes with said universal reagent; (b) contacting said plurality of different analytes labeled with said universal reagent with a collection of distinguishable subpopulations of secondary reagent, each secondary reagent of a given distinguishable subpopulation being designed to interact selectively with a given analyte of said plurality of different analytes, under conditions effective to provide a plurality of complexes, each complex comprised of said distinguishable secondary reagent and said given analyte labeled with said universal reagent; (c) contacting said plurality of complexes with excess reporter reagent, the reporter reagent having an affinity for at least a portion of said universal reagent and further comprised of a detectable label, under conditions effective to provide a plurality of complexes, each complex bearing a detectable label or cleavable group; (d) detecting through said detectable label the existence of a complex and determining through said distinguishable secondary reagent the nature of said given analyte found therein, thereby revealing the identities of the plurality of different analytes present in the fluid sample; and (e) compiling analyte data generated from step (d) in a database. The method may further include a database in a computer readable format.

In further still yet another embodiment of the present invention, a method of generating information useful in the creation of a diagnostic assay for arriving at a likelihood that a subject is suffering from or will later develop one or more diseases is provided, comprising (a) contacting a fluid sample obtained from a subject suffering from or who later develops one or more diseases, which fluid sample is suspected of harboring a plurality of different analytes, with a universal reagent capable of binding to all or substantially all of said plurality of different analytes, under conditions effective to label all or substantially all of said plurality of different analytes with said universal reagent; (b) contacting said plurality of different analytes labeled with said universal reagent with a collection of distinguishable subpopulations of secondary reagent, each secondary reagent of a given distinguishable subpopulation being designed to interact selectively with a given analyte of said plurality of different analytes, under conditions effective to provide a plurality of complexes, each complex comprised of said distinguishable secondary reagent and said given analyte labeled with said universal reagent (c) detecting through said detectable label the existence of a complex and determining through said distinguishable secondary reagent the nature of said given analyte found therein, thereby revealing the identities of the plurality of different analytes present in the fluid sample; (d) repeating steps (a) to (c) using fluid samples from a sufficient number of subjects suffering from or who later develop one or more diseases to provide a database of accumulated analyte data comprising a compilation of analyte data obtained from a statistically significant number of subjects suffering from or who later develop one or more diseases; (e) extracting information from said database, which information is useful in the creation of a diagnostic assay for arriving at a likelihood that a subject is suffering from or will later develop one or more diseases. The method may further comprise selecting a set of given analytes the presence or absence of which appears to correlate with a likelihood that a subject is suffering from or will later develop one or more diseases. In some embodiments, all the subjects chosen for inclusion in the database suffer from or will later develop the same or similar such one or more diseases.

In this respect, before explaining at least one embodiment of the invention in detail, it is to be understood that the invention is not limited in its application to the details of construction and to the arrangements of the components set forth in the following description or illustrated in the drawings. The invention is capable of embodiments in addition to those described and of being practiced and carried out in various ways. Also, it is to be understood that the phraseology and terminology employed herein, as well as the abstract, are for the purpose of description and should not be regarded as limiting.

As such, those skilled in the art will appreciate that the conception upon which this disclosure is based may readily be utilized as a basis for the designing of other structures, methods and systems for carrying out the several purposes of the present invention. It is important, therefore, that the claims be regarded as including such equivalent constructions insofar as they do not depart from the spirit and scope of the present invention.

### BRIEF DESCRIPTION OF THE DRAWINGS

FIG. 1 depicts an assay performed in accordance with one embodiment of the present invention.

FIG. 2 depicts assay performed in accordance with another embodiment of the present invention.

FIG. 3 is a flow diagram of a method for generating a biochemical analyte data database for one or more diseases.

FIG. 4 is a flow diagram of a method for generating a biochemical analyte data profile of a disease.

FIG. 5 is a flow diagram showing a method for designing and generating genetically engineered animals in accordance with one embodiment of the present invention.

### DETAILED DESCRIPTION

In one embodiment of the instant invention, a method of determining the identities and relative quantities of a plurality of different analytes present in a fluid sample is described. The fluid sample to be tested using the instant invention, for example, can include plasma, serum, tears, mucus, saliva, urine, pleural fluid, spinal fluid, gastric fluid, sweat, semen, vaginal secretion, fluid from ulcers and/or other surface eruptions, blisters, abscesses, and/or extracts of tissues, such as biopsies of normal, malignant, and/or suspect tissues.

According to the present invention, assay components and methods are provided for the measurement of analytes, defined broadly herein to encompass all biomolecules including, but not limited to, proteins, DNA and RNA fragments, and lipids. More specifically, the analytes of interest for these bioassays include, for example, amino acids, antigens, antibodies, peptides, modified oligopeptides, polypeptides, proteins, glycoproteins, lipoproteins, and/or enzymes. The enzymatic analytes includes, for example, proteases, glycosidases, nucleotidases, oxidoreductases, hydrolases, esterases, convertases, ligases, transferases, phosphorylases, lyases, lipases, peptidases, dehydrogenases, oxidases, phospholipases, invertases, aldolases, transaminases, synthetases, and/or phosphotases.

The antigenic analytes, for example, includes bacterial, viral, fungal, mycoplasmal, ridkettsial, chlamydial, and/or protozoal antigens. Alternatively, the antigens, for example, include antigens borne by pathogenic agents responsible for a sexually transmitted disease, antigens borne by pathogenic agents responsible for a pulmonary disorder, and/or antigens borne by pathogenic agents responsible for gastrointestinal disorder.

The inventive technology can improve the speed and sensitivity of determining the identities and relative quantities of a plurality of different analytes substantially simultaneously while reducing the cost of performing diagnostic and genetic assays. In one embodiment, a multiplexed assay in accordance with the invention obviates the need for two different reagents with specificity for a single analyte. Broadly, development of a multiplexed assay for use in accordance with the invention can be divided into three phases: (1) universal reagenting of the analytes, (2) specific labeling of predetermined analytes of interest, and (3) identification and quantification of the analytes of interest. The three general steps will now be discussed in greater detail.

Universal Reagent

In one aspect of the invention, a universal reagent is provided for non-specifically binding to a substantial fraction of analytes in a sample. A substantial fraction is greater than about 50% of the analytes, preferably greater than about 75% of the analytes, and more preferably, greater than about 95% of the analytes in a given sample. By “non-specifically,” it is meant that the universal reagent is capable of binding to any available and exposed functional group that is common to a class of biomolecules. For example, a universal reagent in accordance with the present invention may non-specifically bind to nitrogen or sulfur containing molecules, which likely encompasses many, if not all, nucleic acids and proteins.

Preferably, in some embodiments, the universal reagent is a metal complex. More preferably, the universal reagent is a platinum (Pt) metal complex as described in U.S. Pat. Nos. 5,714,327 and 5,985,566, which are incorporated by reference herein in their entirety. Platinum-based metal complexes for labeling of biomolecules can be obtained from manufacturers such as Kreatech Biotechnology B.V. in The Netherlands.

The reaction of a universal reagenting compound with a biomolecule is generally controllable and substantially irreversible, permitting the stable and detectable labeling of a variety of biological compounds. The reaction of the labeling compound with a target molecule may involve the displacement of a leaving group bound to Pt to be replaced with a single linkage to the target molecule. Accordingly, the displaceable leaving group may be any group which can be displaced by a biomolecule when the labeling compound and the biomolecule react with one another.

Leaving groups suitable for use in the compounds of the invention would include any group which would be replaced in favor of a bio-organic molecule of interest under appropriate conditions. For example, suitable leaving groups for the labeling of a nucleic acid would include groups which would permit a bond between the Pt atom and the protein to be formed under appropriate conditions. The selection of leaving groups, therefore, is within the discretion of the artisan with respect to the system in which the platinum compounds are to be employed and the target molecules which are to be labeled.

As leaving ligands, (CH₃)₂SO, H₂O and Cl are especially suitable. Other leaving ligands suitable for use include halogens, SO₃²⁻, NO₃⁻, PO₄³⁻, CO₃²⁻, and analogs like ethylnitrate; phosphonates, carboxylates, oxalates, citrates and derivatives thereof; H₂O, ROH and RO⁻, in which R is an organic residual group and substituted sulfoxides R¹R²SO, in which R¹ and R², whether or not identical to each other, represent organic residual groups.

A universal reagent can further comprise any number of suitable detectable labels or marker compounds known in the art. For the purposes of the present invention, the detectable label should provide a signal related to the presence of analyte in the sample which results in the detection of electromagnetic radiation, particularly light in the ultra-violet, visible or infrared range. The universal reagent may be detected by directly or indirectly methods such as those known in the art. Direct methods of labeling may be radioactive labels, colloidal dye substances, fluorochromes, chromophores, etc., which are provided directly bound to the metal of the universal reagent and are “directly” detectable. Alternatively, indirect methods of labeling may include specific binding pair components (e.g., avidin, streptavidin, biotin, biocytin, iminobiotin), in which a “reporter reagent” provides the detectable label. Indirect methods of labeling also include marker enzymes (which need reaction with a substrate to be detected).

By having fluorescent detectable labels, such as fluorescent particles, fluorescent conjugated antibodies, or the like, the sample may be irradiated with light absorbed by the fluorescers and the emitted light measured by light measuring devices. In embodiments where the fluorochromes are preferred detectable labels, fluorescent groups may be selected from among fluoresceins, eosins, trisulfonylpyrenes, rhodamines, digoxigenins, 4,4-difluoro-4-bora-3a,4a-diaza-s-indacenes, and derivatives thereof. Especially preferred detectable labels include fluorescein isothiocyanate (FITC) or tetramethyl rhodamine isothiocyanate (TRITC).

One or more of these labels may be attached to the platinum compounds either directly or through spacer arms leaving the potential for multiple detections in one assay. Spacer arms are particularly desirable in cases in which steric hindrances due to molecular dimension or interaction would otherwise inhibit or prevent binding.

Specific Reagent

In another aspect of the invention, a collection of distinguishable subpopulations of a secondary, or “specific” reagent, is provided for specifically binding to predetermined residues of a substantial fraction of the analytes labeled with universal reagent. The specific reagent, such as antibodies, antigens, peptides, proteins, enzymes and/or nucleic acid probes, provides specific sites for predetermined analytes in a multiplexed assay. Each secondary reagent may be selected to react, optionally uniquely, to an analyte of interest in a biological sample.

The secondary reagent, in turn, may optionally be conjugated to a substantially solid support, such as, for example, a microchip or a microsphere. The secondary reagent and/or the solid support is further coupled to a direct or indirect reporter label such as those described above for universal reagents. Alternatively, a solid support may be a chip, preferably a planar microchip, with a spatial arrangement of reagents on a two-dimensional surface. In some embodiments, commercially available microchips may be used, such as those available from Affymetrix, Inc.

Microspheres are alternately termed microparticles, beads, polystyrene beads, microbeads, latex particles, latex beads. In cases where the microspheres are coated or supplied with one or more flourophores or dyes, the microspheres are also called fluorescent beads, fluorescent particles, colored particles and colored beads. Beads suitable for use as a starting material in accordance with the invention are generally known in the art and may be obtained from manufacturers such as Spherotech and Molecular Probes. Methods of imbuing color to the beads are similarly also known in the art and include attachment to the microsphere surface by covalent bonding or adsorption.

Microspheres described can be obtained from manufacturers, such as Luminex Corp. of Austin, Tex. Illustrative microspheres and methods of manufacturing same are, for example, found in U.S. Pat. No. 6,268,222 to Mark B. Chandler and Don J. Chandler, entitled “Microparticles attached to nanoparticles labeled with flourescent dye,” and in U.S. Pat. No. 6,632,526 to Don J. Chandler, Van S. Chandler, and Beth Lambert, entitled “Precision fluorescently dyed particles and methods of making and using same.” Both patents are incorporated herein by reference in their entirety.

Assay Procedure

Assays can be carried out in accordance with the various protocols. In accordance with the subject invention, broadly, a sample is contacted with a plurality of predetermined reagents and various operations may be carried out, such as the addition of miscellaneous reagents, incubations, washings, and the like. The final result of the assays will be the change in the amount of a product, which absorbs or produces a dye, or light, either by light absorption or by light emission in relation to the presence or amount of the analyte of interest. Usually, this is a result of formation of a specific binding complex between complementary members of a specific binding pair, where one of the members may serve as a bridge to form a sandwich, or there may be a single complex, or complexes may be bound to complex binding proteins.

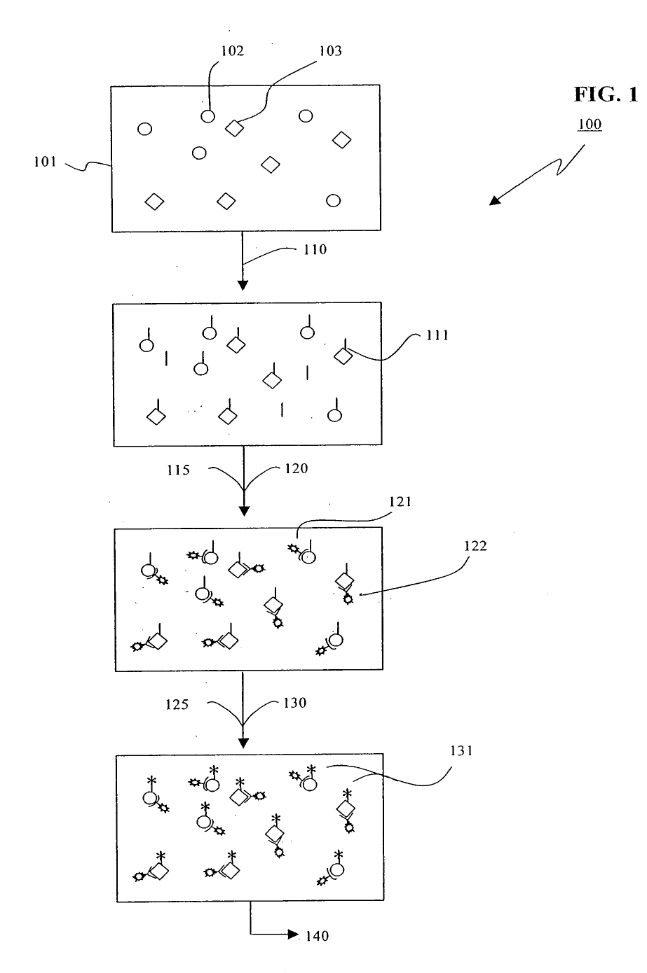

An illustrative general method of operation of an assay procedure 100 according to the invention is shown, by way of example, in FIG. 1, wherein a universal reagent is detected by an indirect methodology. In step 110, an excess of universal reagent 111 is contacted with the biological fluid sample 101 which is suspected of harboring a plurality of different analytes 102, 103 containing residues reactive with the universal reagent under conditions that will permit analyte-universal reagent interaction.

Universal reagent should be introduced in an amount to at least saturate any and all possible binding sites. The amount will depend from sample to sample, but may be determined by trial and error by known methods appreciated by a skilled artisan. Preferably, universal reagent is added in greater than a 2:1 ratio relative to binding sites available, more preferably, greater than a 5:1 ratio, and even more preferably, greater than a 10:1 ratio. Solely in the interest of simplicity, one universal reagent 111 has been depicted to bind to different analytes 102, 103. However, as mentioned above, universal reagents of the present invention may bind to an analyte at a plurality of sites, and in some instances, none at all.

Prior to step 120, an optional wash step 115 may be incorporated. Wash step 115 may be designed to remove a substantial fraction of unbound universal reagent 111. The excess universal reagent 111 could interfere with subsequent steps of the assay procedure 100. Thus, the removal of unbound universal reagent 111 may improve sensitivity of the assay procedure 100.

In step 120, a collection of distinguishable subpopulations of secondary reagents 121, 122 are added to the sample fluid 101 harboring a plurality of different anlaytes labeled with the universal reagent 111. Preferably, the secondary reagents 121, 122 are fluorescent microspheres and include a pooled population of classes or subsets of microspheres. Each subset of microspheres includes one, two, or more microspheres. Advantageously, a plurality of microspheres per subset are used, for example, up to 1000 or more. Each microsphere in a respective subset of microspheres includes one, two, three, four, five or more classification parameters. For example, one classification parameter may include a forward light scatter parameter, and another may include a side light scatter parameter. The classification parameters of each microsphere advantageously includes one, two, three, or more standard fluorochromes or fluorescent dyes.

As will be appreciated by one of ordinary skill in the art from the teachings herein, the amount of secondary reagent 121, 122 will depend on the relative amount of potential targets present in the sample 101. That is, samples with higher concentration of protein will likely require a larger amount of secondary reagent 121, 122. In any case, as with the universal reagent 111, a molar excess of secondary reagent 121, 122 to analyte 102, 103 is preferred.

Prior to step 130, an optional wash step 125 may be performed. As the secondary reagents 121, 122 are fluorescently labeled, removal of unbound secondary reagent 121, 122 can reduce background fluorescence and thereby improve sensitivity of the bioassay 100.

In step 130, the sample fluid 101 harboring a plurality of complexes comprising distinguishable secondary reagent 121, 122, analyte 102, 103, and universal reagent 111, are contacted with a molar excess of a reporter reagent 131. A plurality of complexes are thus formed, each complex comprising a distinguishable secondary reagent and a analyte labeled with a universal reagent with reporter reagent. Thus, a universal reagent as defined herein has two functions: the capacity to react with the analyte and the capacity to provide a detectable signal, which is distinct from the fluorescence signal of the secondary reagent, or microsphere, of the invention.

The reporter reagent should have an affinity for at least a portion of the universal reagent 111 and further comprise a detectable label. For example, the universal reagent 111 could comprise a biotin marker. In such case, an appropriate reporter reagent might be avidin or streptavidin bearing a detectable label such as a fluorochrome. As with the prior steps, a wash step may be performed prior to step 140.

In step 140, the identity and quantity of the analytes 102, 103 are determined substantially simultaneously to the running step. Preferably, where fluorescent labels or microspheres are involved, detection of the labels is carried out in a flow cytometer. The existence of a complex is determined by detecting the presence of a detectable label and the identity or nature of the of a given analyte in a complex is determined by detecting the predetermined secondary reagent. The nature of a given analyte may include the class or subclass of molecule (e.g., an enzyme or kinase, respectively); whereas the identity may include the exact molecule (e.g., hexokinase).

An ordinary flow cytometer is capable of analyzing spectral properties (fluorescent signals) of up to 20,000 particles per second and can provide reliable quantitative data in real-time scale. A suitable flow cytometer for use with this invention is the Coulter Elite-ESP flow cytometer, or FACScan flow cytometer available from Beckman Coulter, Inc., Fullerton, Calif. Also suitable is the MOFLO flow cytometer available from Cytomation, Inc., Fort Collins, Colo.

Acceptable alternative embodiments of the method of operation are optionally found in U.S. Pat. No. 6,139,800 to Mark B. Chandler, and U.S. Pat. No. 6,449,562, to Van S. Chandler, Jerrold R. Fulton, and Mark B. Chandler, both references being incorporated herein by reference in their entirety. In addition to flow cytometry, for example, a centrifuge may be used as the instrument to separate and classify the microparticles. A suitable system is that described in U.S. Pat. No. 5,926,387, incorporated herein by reference.

In addition to flow cytometry and centrifugation, a free-flow electrophoresis apparatus may be used as the instrument to separate and classify the microparticles. A suitable system is that described in U.S. Pat. No. 4,310,408, incorporated herein by reference.

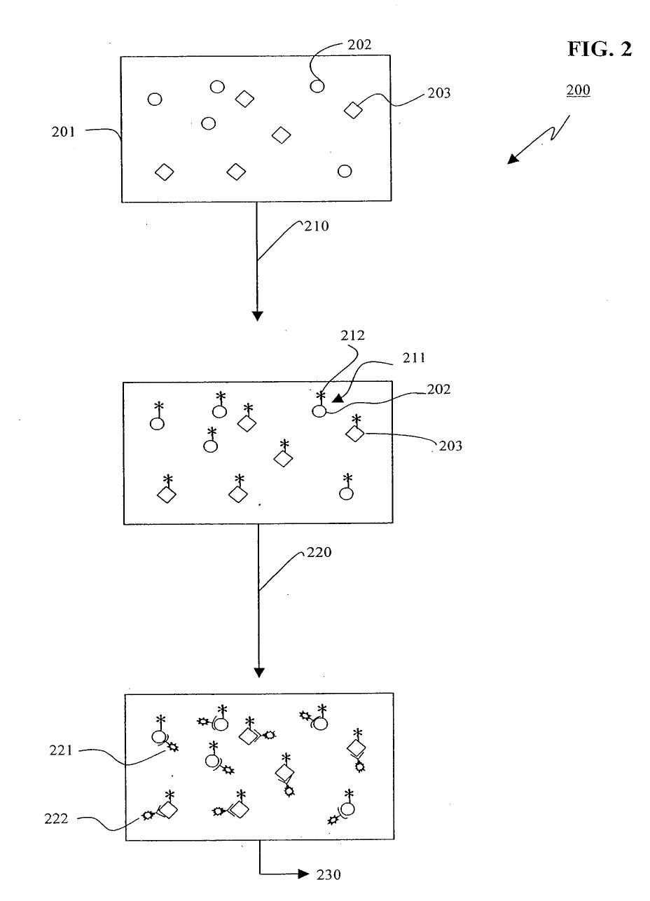

Another illustrative general method of operation of an assay procedure 200 according to the invention is shown, by way of example, in FIG. 2, wherein a universal reagent with a directly bound detectable label is used. In step 210, an excess of a universal reagent 211 bearing a detectable label 212 is contacted with a biological fluid sample 201 which is suspected of harboring a plurality of different analytes 202, 203 containing residues reactive with the universal reagent under conditions that will permit analyte-universal reagent interaction.

In step 220, a collection of distinguishable and predetermined secondary reagents 221, 222 are added to the sample fluid 101 harboring a plurality of different analytes 202, 203 labeled with the universal reagent 211. Preferably, the secondary reagents 221, 222 are fluorescent microspheres and include a pooled population of classes or subsets of microspheres as described above.

In step 230, the identity and quantity of the analytes 202, 203 are determined by any one of methods described above. Preferably, where fluorescent labels or microspheres are involved, detection of the labels is carried out in a flow cytometer. The existence of a complex is determined by detecting the presence of a universal reagent 211 and the identity or nature of the of a given analyte in a complex is determined by detecting the predetermined secondary reagent 221, 222.

As will be appreciated by one of ordinary skill in the art from the teachings herein, the amount of universal reagent 211 and secondary reagent 121, 122 can depend on the relative amount of potential targets present in the sample 201. That is, samples with higher concentration of protein will likely require a larger amount of universal and secondary reagents. It will be also understood from the teachings herein, that methods of amplifying signals from the universal and/or secondary reagents may be incorporated into the inventive method. By way of example, any known enzymatic, chemiluminescent or fluorescent, or amino dextran method of enhancing or amplifying a signal may be performed at any given step during the assay.

Prior to steps 220 and 230, an optional wash step may be incorporated. Washes may be designed to remove a substantial fraction of unbound universal reagent 211 and secondary reagents 221, 222. The unbound excess of either reagent could interfere with subsequent steps of the assay procedure 200. Thus, the removal of unbound reagent may improve sensitivity of the assay procedure by at least reducing background fluorescence.

The data that is generated from methods according to the instant invention the recording, storing, processing, and display of data generated in the assay procedures are not limited to any one embodiment. For example, analyte data may be recorded in analog or digital format; stored in RAM, hard drive, magnetic tape, or paper copy; processed manually, by computers, or other machines; and displayed on a printer, computer monitor, or otherwise, by way of examples. In a preferred embodiment, data collected by flow cytometer is digitally recorded with a computer and stored in a database.

As those of ordinary skill in the art will recognize from the teachings herein, the invention has numerous applications in diagnostic assay techniques. Generally, the present invention can provide bioassays including, for example, immunoassays, complex genetic analyses, and enzymatic assays. To this extent and others U.S. Pat. No. 5,981,180 to Van S. Chandler, Jerrold R. Fulton, and Mark B. Chandler and U.S. Provisional Application Ser. No. 60/510,093 also incorporated herein by reference in their entirety.

Reagents may be prepared, for example, so as to detect or screen for any of a number of sample characteristics, pathological conditions, or reactants in fluids. Reagents may be designed, for example, to detect antigens or antibodies associated with any of a number of infectious agents including (without limitation, bacteria, viruses, fungi, mycoplasma, rickettsia, chlamydia, and protozoa), to assay for autoantibodies associated with autoimmune disease, to assay for agents of sexually transmitted disease, or to assay for analytes associated with pulmonary disorders, gastrointestinal disorders, cardiovascular disorders, and the like. Similarly, the reagent may be designed to detect any of a number of substances of abuse, environmental substances, or substances of veterinary importance.

It will be apparent from the teachings herein that the present invention can be used in many applications. In one embodiment, the assays in accordance with the present invention provide one or more methods of generating and using electronic data comprising biochemical analyte data. The biochemical data may comprise, for example, measurements taken of a plurality of different analytes present in a specimen (e.g., blood); one or more measurements taken from different specimens (e.g., blood and urine) from a single subject; or one or more measurements taken from one or more specimens from multiple subjects from a sample of a population. Therefore, any data comprising a plurality of measurements can be used to diagnose and classify a plurality of diseases.

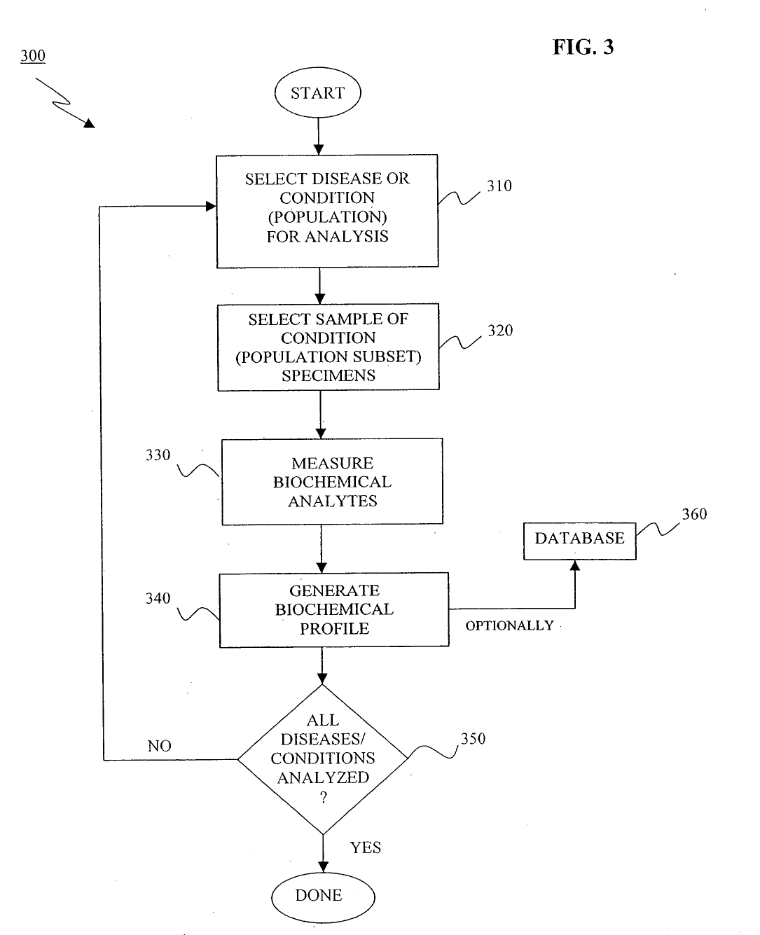

Referring now to FIG. 3, there is a flow diagram of a method 300 for generating a biochemical analyte profile of a disease for a given population of animals. “Animals” of the present invention comprise any of living multicellular organisms that may be of potential interest for scientific or medical investigation. Preferably, “animal” refers to vertebrates including, but not limited to humans, primates, rabbits, and rodents, such as, for example, mice, guinea pigs, and rats. The method 300 may be repeated to generate a database of biochemical data of a single disease (e.g., diabetes) from different populations (e.g., teenage children or adults over age 65) and also of different diseases (e.g., diabetes or asthma) in a single population (e.g., teenage children).

In step 310, a disease is selected for analysis. In other words, a population having a common disease or set of characteristics is selected. The disease selected may be studied from the entire population sharing in common the disease, for example, diabetes. Alternatively, the disease selected for study may be further limited to a population having a common age bracket, gender, species, or in the case with humans, race. Thus, for example, the disease selected for analysis may correspond to a population of diabetic patients associated with Caucasian males between ages 35-65 or a population of obese female mice. It should be understood that any population selected for analysis of a disease can correspond to either a control (i.e. “normal”) group or one with a disease (i.e. “abnormal”).

In step 320, a sample of subjects is selected from the population selected for analysis in step 310. Preferably, the sample includes a number of subjects sufficient to permit a statistically significant analysis of the population as a whole. Thus, preferably, the sample includes a number of subjects such that the biochemical analyte data generated from the sample corresponds to a statistically significant representation of those biochemical analytes for the population as a whole.

Referring still to FIG. 3, in step 330, a plurality of biochemical analytes are measured from the sample 320. The measurements are representative of exposure of a biological specimens from a sample of subjects of a population to a plurality of biological assays. In generating the biochemical data of the present invention, many types of test specimens from sample 320 can be used.

In step 340 of FIG. 3, the biochemical data collected in step 330 is electronically processed to generate a biochemical data profile of the disease. Preferably, in some embodiments, computational software may be used for mining and pooling data from multiple specimens. Such software permits the incorporation of relevant information, even from other domains, such as medical history information, or phenotype information, in generating a biochemical data profile. Once generated, the biochemical data from step 340 may be optionally stored in a database 360 or programmed into a microprocessor to be used for correlations, such as, for example, with data from a test subject.

As indicated in step 350, the assay may be repeated for each and any population of interest. All of the biochemical data associated with the population or populations of interest and described above may be stored in the database 360, and may optionally include correlation values as discussed above for each population of interest. By repeating this process for each population of interest, the present invention may be optionally used to generate a database 360 which includes biochemical analyte data for many different diseases. Alternatively, a single statistically significant representative data for a given population 320 may be stored electronically or embedded into a software program for comparison or correlation with an data gathered from a test subject.

In this manner, scientific investigators and/or medical practitioners may gather biochemical data of a patient and assess the patient's disease based on an data with quantitative and/or qualitative data of the analytes themselves. For example, a specimen from each subject from a sample with a disease may be analyzed and compared and correlated with a comprehensive database of biochemical data of disease states to determine the likelihood of a given disease being present.

“Correlations” comprise, for example, comparisons between selected pairs of data. In one example, selected pairs of biochemical data from different populations of cancer patients (e.g., prostate cancer or breast cancer) can be correlated with each other. Such correlations may reveal similarities or differences between cancer types that may aid in the identification and study of a respective disease. Similarly, selected pair of biochemical data from different diabetic populations (e.g., ages 13-18 or ages 55-75) can be correlated with each other, which may reveal information regarding the progression of a disease. It will be understood by those skilled in the art that correlation other than those enumerated above may be made and stored in step 340, and that the use of such other correlations are within the scope of the present invention. For example, selected biochemical data from a diabetic population may be correlated with biochemical data from obese patients.

These types of correlations can further provide information relating to the prognosis of a patient. In fact, it is expected that the present invention may enable the detection of disease, such as, for example, cancer, at times earlier than is now possible with conventional technologies, particularly in cases where diseases are manifested in changes in analytes that can be detected by biochemical methods and represented by biochemical imaging. Similarly, the early onset of heart disease and diabetes can be detected in time to allow pre-symptomatic intervention.

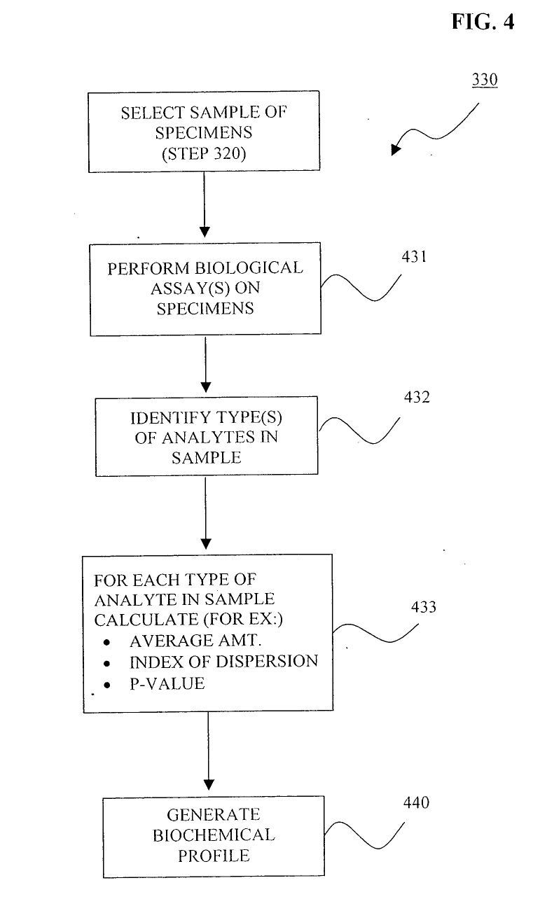

Referring now to FIG. 4, there is shown a flow diagram of the step 330 for profiling the subset 320 of a population of subjects with shared characteristics in order to generate a biochemical analyte data profile that represents characteristics of a disease associated with the population. In step 431, at least one biochemical assay (preferably, a plurality, and more preferably at least 50) is applied to each specimen from each subject from the sample selected in step 420. The biochemical assay(s) that may be used for a given specimen include, for example, total protein content, total nucleic acid content, total lipid content assays, and/or their respective individual elements such as specific proteins, specific nucleic acid, and specific lipid content assays. In one embodiment, one or more assays are applied to a plurality of specimens in each subject or disease studied.

In step 432, the biochemical data from step 431 is analyzed in order to identify types of biochemical analytes that are present in the sample. The types of analytes identified for analysis preferably correspond to the types of analytes that distinguish the disease population of interest from other control populations. For example, where diseases of the immune system are known in the sample of the population, cytokines may be particularly examined. In step 433, three exemplary values are preferably determined for each type of analyte that was identified in step 432. More particularly, for each identified type of analyte, the following values are determined in step 433: (i) the average amount of the particular type of analyte in the sample, (ii) an index of dispersion associated with the measured average amount of the particular type of analyte, and (iii) the p-value associated with the measurement.

Referring still to step 433, for each identified type of analyte, the average amount of the particular type of analyte in the sample of the population and the index of dispersion associated with the measured average amount of the particular type of analyte are determined by first analyzing the biochemical assay information corresponding to each sample of the population in order to determine the average amount of the particular type of analyte in each such specimen. By performing such an analysis on each specimen in the sample, a distribution of analyte values for the particular type of analyte may then be obtained.

An average amount index representative of an average amount of the particular type of analyte in the population is then calculated by taking the statistical average of this distribution. Similarly, a standard deviation about the average amount of the particular type of analyte in the population is calculated by, for example, taking the standard deviation, standard error, or standard error of the mean of the distribution of analyte amount values obtained for the particular type of analyte from the sample.

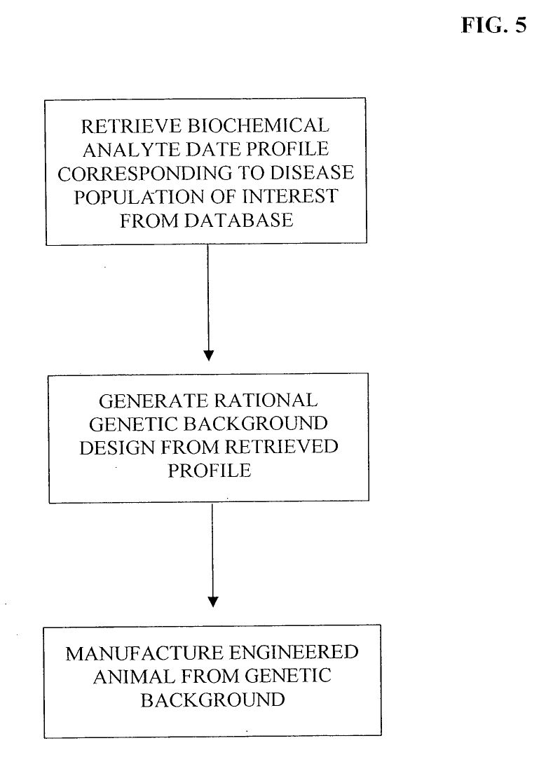

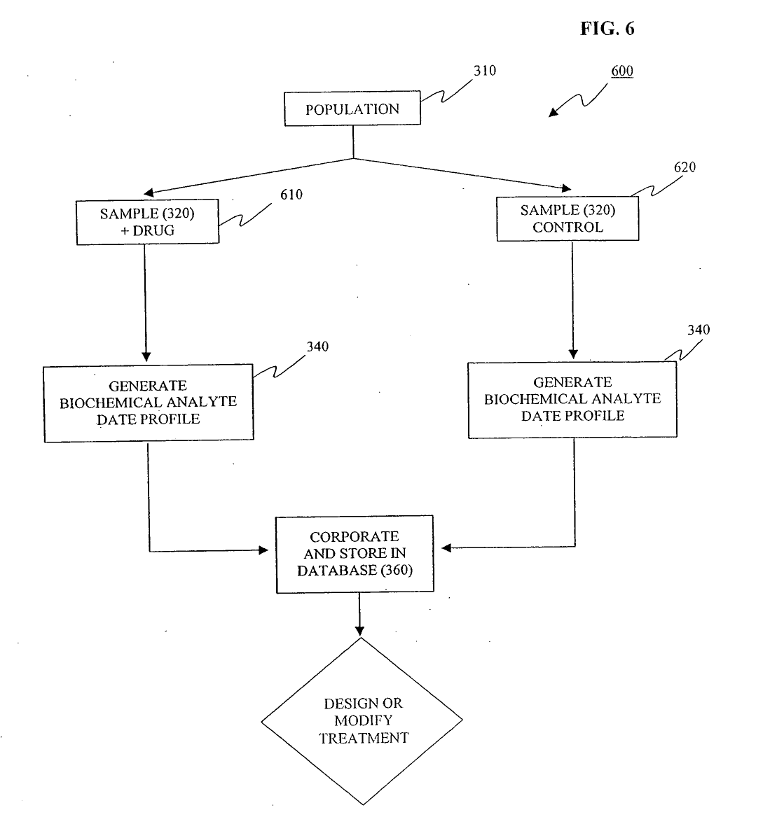

The biochemical data associated with each disease studied may also be processed to collectively represent a “blueprint” of the disease in the population 320 and may be used, inter alia, to rationally design and then manufacture animal models corresponding to the diseased population. For example, as depicted in a flow chart in FIG. 5, a model designed for a given disease may include animals that have been genetically engineered to include and/or exclude genes and protein factors that yield an animal with a biochemical analyte data profile similar to that observed in the human disease population. Thus, in one particular example, the leptin deficient mice may be generated to reflect leptin deficiency commonly associated with obesity in mammals. Alternatively, biochemical data taken from animals genetically engineered to mimic a human disease, may also be used for comparison with biochemical data of humans with the respective disease. In this manner, biochemical data of a disease may be used to validate the use of an animal model to study the disease.

The inventive method can also be used to study the underlying biochemical basis for side effects of administering drugs. FIG. 6 is a flow chart illustrating the steps 600 that may be followed in accordance with one embodiment of the instant method to improve drug safety and efficacy or therapeutic treatment of an animal with a disease based on a biochemical analyte profile.

Using the instant inventive method, a sample of a population sharing a common disease could be divided into two subpopulations 610, 620, one treated with a drug of interest and one without same. Biological specimens, preferably blood and preferable from a statistically representative sample size, could be donated and analyzed of its biochemical analytes. A biochemical data profile 340 can then be generated from the data gathered from each of the sample populations 610, 620. Such differences may be representative of biochemical manifestations of drug safety concerns, drug efficacy, and generally, drug side effects. Based on the differences in analyte images between subpopulations 610, 620, a new or modified treatment may be developed to counter some or all of the side effects and improve drug performance and efficacy.

In some embodiments in accordance with the invention, the assays described herein may be provided for various diagnostic, analytic, and industrial applications known in the art. It is a still further object of the invention to provide a kit suitable for use in detection of the analytes of interest. Preferably, this kit contains a series of microparticles with attached nanoparticles having a distinct fluorescent signal and also an analytical reactant capable of specifically binding with one of analytes of interest. The kit also contains a universal reagent comprising a reagent, which binds to the same analyte as the analytical reagent and also this kit may contain a fluorescent label, a competitor molecule, a reference material, and other ingredients that are accepted as standard reagents such as a wash buffer, necessary plasticware, etc.

The inventive method allows the detection of a plurality of analytes simultaneously during a single flow cytometric processing step. Benefits of the inventive multiplex assay method include increased speed and reduced cost to analyze a clinical sample.

While the invention has been described in connection with specific embodiments thereof, it will be understood that it is capable of further modifications and this application is intended to cover any variations, uses, or alterations of the invention following. In general, the principles of the invention and including such departures from the present disclosure as come within known or customary practice within the art to which the invention pertains and as may be applied to the essential features hereinbefore set forth and as follows in the scope of the appended claims.

1. A method of revealing the identities of a plurality of different analytes present in a fluid sample comprising:
(a) contacting a fluid sample, which is suspected of harboring a plurality of different analytes, with a universal reagent capable of binding to all or substantially all of said plurality of different analytes, under conditions effective to label all or substantially all of said plurality of different analytes with said universal reagent;
(b) contacting said plurality of different analytes labeled with said universal reagent with a collection of distinguishable subpopulations of secondary reagent, each secondary reagent of a given distinguishable subpopulation being designed to interact selectively with a given analyte of said plurality of different analytes, under conditions effective to provide a plurality of complexes, each complex comprised of said distinguishable secondary reagent and said given analyte labeled with said universal reagent; and
(c) detecting through said universal reagent the existence of a complex and determining through said distinguishable secondary reagent the nature of said given analyte found therein, thereby revealing the identities of the plurality of different analytes present in the fluid sample.

2. The method of claim 1 in which the quantities of the plurality of different analytes in the fluid sample is revealed.

3. The method of claim 1 in which said universal reagent comprises one or more analyte binding moieties and one or more detectable labels.

4. The method of claim 3 in which the detectable label comprises a fluorescent group, a radioactive atom, or a combination thereof.

5. The method of claim 3 in which the analyte binding moiety is a protein, nucleic acid, or a lipid.

6. The method of claim 1 in which said universal reagent comprises a metal complex.

7. The method of claim 6 in which said metal complex comprises a platinum metal complex labeled with a fluorescent group.

8. The method of claim 6 in which said metal complex comprises a metalloporphyrin or platinum metal complex including (i) biotin or a biotin-containing moiety and (ii) a leaving group.

9. The method of claim 1 in which said collection of distinguishable subpopulations of secondary reagent comprises planar substrates, magnetic beads, or fluorescent microspheres.

10. The method of claim 1 in which said secondary reagent comprises polypeptides, polynucleotides, or combinations thereof.

11. The method of claim 10 in which said polypeptides comprise antibodies or the binding regions thereof.

12. The method of claim 9 in which said fluorescent microspheres form distinguishable subpopulations through distinguishable combinations of two or more fluorophores.

13. The method of claim 1 in which step (c) is carried out in a flow analyzer.

14. The method of claim 4 in which the universal reagent and the secondary reagent each exhibit one or more characteristic fluorescence emission classification parameters.

15. The method of claim 14 in which the universal reagents differ from the secondary reagents in an intensity of at least one fluorescence emission classification parameter.

16. The method of claim 1 in which said given analyte comprises a hormone, antigen, cytokine, antibody, or oligopeptide.

17. The method of claim 1 in which said given analyte comprises genomic DNA, cDNA, oligonucleotides, RNA, or RNA nucleotides.

18. The method of claim 1 in which the fluid sample is selected from the group consisting of plasma, serum, tears, mucus, saliva, urine, pleural fluid, spinal fluid, gastric fluid, sweat, semen, vaginal secretion, broncioalveolar lavage, sputum, breath condensate, peritoneal fluid, joint fluid, fluid from ulcers and other surface eruptions, blisters, abscesses, and extracts of tissues including biopsies of normal, malignant, or suspect tissues.

19. The method of claim 14 further comprising amplifying the fluorescence emission of the universal reagent or the secondary reagent.

20. The method of claim 1 in which the fluid sample comprises cell lysate.

21. The method of claim 1 in which the fluid sample is obtained from a mammal.

22. The method of claim 21 in which the mammal is a human.

23. The method of claim 1 further comprising removing unbound secondary reagent prior to performing step (c).

24. The method of claim 1 in which said universal reagent or said secondary reagent is coupled to a solid support.

25. The method of claim 24 in which the solid support comprises a microsphere, microchip, biochip, or planar array.

26. A method of revealing the identities of a plurality of different analytes present in a fluid sample comprising:
(a) contacting a fluid sample, which is suspected of harboring a plurality of different analytes, with a universal reagent capable of binding to all or substantially all of said plurality of different analytes, under conditions effective to label all or substantially all of said plurality of different analytes with said universal reagent;
(b) contacting said plurality of different analytes labeled with said universal reagent with a collection of distinguishable subpopulations of secondary reagent, each secondary reagent of a given distinguishable subpopulation being designed to interact selectively with a given analyte of said plurality of different analytes, under conditions effective to provide a plurality of complexes, each complex comprised of said distinguishable secondary reagent and said given analyte labeled with said universal reagent;
(c) contacting said plurality of complexes with excess reporter reagent, the reporter reagent having an affinity for at least a portion of said universal reagent and further comprised of a detectable label or cleavable group, under conditions effective to provide a plurality of complexes, each complex bearing a detectable label or cleavable group; and
(d) detecting through said detectable label the existence of a complex and determining through said distinguishable secondary reagent the nature of said given analyte found therein, thereby revealing the identities of the plurality of different analytes present in the fluid sample.

27. The method of claim 26 in which said universal reagent comprises a metal complex.

28. The method of claim 27 in which said metal complex comprises a platinum metal complex including biotin or a biotin-containing moiety.

29. The method of claim 28 in which the reporter reagent is avidin bound to a detectable label.

30. The method of claim 29 in which said detectable label gives rise to fluorescence, chemiluminescence, or a color change.

31. The method of claim 26, further comprising removing unbound reporter reagent prior performing step (d).

32. A method of arriving at a likelihood that a subject is suffering from or will later develop one or more diseases, comprising:
(a) contacting a fluid sample, which is suspected of harboring a plurality of different analytes, with a universal reagent capable of binding to all or substantially all of said plurality of different analytes, under conditions effective to label all or substantially all of said plurality of different analytes with said universal reagent;
(b) contacting said plurality of different analytes labeled with said universal reagent with a collection of distinguishable subpopulations of secondary reagent, each secondary reagent of a given distinguishable subpopulation being designed to interact selectively with a given analyte of said plurality of different analytes, under conditions effective to provide a plurality of complexes, each complex comprised of said distinguishable secondary reagent and said given analyte labeled with said universal reagent;
(c) contacting said plurality of complexes with excess reporter reagent, the reporter reagent having an affinity for at least a portion of said universal reagent and further comprised of a detectable label or cleavable group, under conditions effective to provide a plurality of complexes, each complex bearing a detectable label or cleavable group;
(d) detecting through said detectable label the existence of a complex and determining through said distinguishable secondary reagent the nature of said given analyte found therein, thereby revealing the identities and quantities of the plurality of different analytes present in the fluid sample;
(e) comparing analyte data generated from step (d) with a database of accumulated analyte data, which accumulated analyte date comprises a compilation of analyte data obtained from a statistically significant number of subjects suffering from or who later develop one or more diseases;
(f) optionally arriving at a likelihood that such subject is suffering from or will later develop one or more diseases based, at least in part, on the results of said comparison.

33. A method of generating an animal model of a disease comprising:
(a) contacting a fluid sample, which is suspected of harboring a plurality of different analytes, with a universal reagent capable of binding to all or substantially all of said plurality of different analytes, under conditions effective to label all or substantially all of said plurality of different analytes with said universal reagent;
(b) contacting said plurality of different analytes labeled with said universal reagent with a collection of distinguishable subpopulations of secondary reagent, each secondary reagent of a given distinguishable subpopulation being designed to interact selectively with a given analyte of said plurality of different analytes, under conditions effective to provide a plurality of complexes, each complex comprised of said distinguishable secondary reagent and said given analyte labeled with said universal reagent;
(c) contacting said plurality of complexes with excess reporter reagent, the reporter reagent having an affinity for at least a portion of said universal reagent and further comprised of a detectable label or cleavable group, under conditions effective to provide a plurality of complexes, each complex bearing a detectable label or cleavable group;
(d) detecting through said detectable label the existence of a complex and determining through said distinguishable secondary reagent the nature of said given analyte found therein, thereby revealing the identities of the plurality of different analytes present in the fluid sample;
(e) determining a relationship between one or more given analytes and the disease of the population of subjects whose accumulated biochemical analyte data share similar features; and
(f) genetically manipulating an animal to express the one or more given analytes related to the disease of interest.

34. The method of claim 33 in which the animal is a rodent.

35. A method of determining the effects of drug administration on a subject expressing one or more given analytes comprising:
(a) contacting fluid samples taken from a subject both before and after administration of a drug, which samples are suspected of harboring a plurality of different analytes, with a universal reagent capable of binding to all or substantially all of said plurality of different analytes, under conditions effective to label all or substantially all of said plurality of different analytes with said universal reagent;
(b) contacting said plurality of different analytes labeled with said universal reagent with a collection of distinguishable subpopulations of secondary reagent, each secondary reagent of a given distinguishable subpopulation being designed to interact selectively with a given analyte of said plurality of different analytes, under conditions effective to provide a plurality of complexes, each complex comprised of said distinguishable secondary reagent and said given analyte labeled with said universal reagent;
(c) contacting said plurality of complexes with excess reporter reagent, the reporter reagent having an affinity for at least a portion of said universal reagent and further comprised of a detectable label or cleavable group, under conditions effective to provide a plurality of complexes, each complex bearing a detectable label or cleavable group;
(d) detecting through said detectable label the existence of a complex and determining through said distinguishable secondary reagent the nature of said given analyte found therein, thereby revealing the identities of the plurality of different analytes present in the fluid samples; and
(e) comparing the analyte data generated from the sample taken before administration of the drug with the analyte data generated from the sample taken after administration of the drug to provide the effect(s), if any, of said drug administration on the analyte data expressed by the subject.

36. A computer implemented method for generating information on a disease comprising:
(a) contacting a fluid sample, which is suspected of harboring a plurality of different analytes, with a universal reagent capable of binding to all or substantially all of said plurality of different analytes, under conditions effective to label all or substantially all of said plurality of different analytes with said universal reagent;
(b) contacting said plurality of different analytes labeled with said universal reagent with a collection of distinguishable subpopulations of secondary reagent, each secondary reagent of a given distinguishable subpopulation being designed to interact selectively with a given analyte of said plurality of different analytes, under conditions effective to provide a plurality of complexes, each complex comprised of said distinguishable secondary reagent and said given analyte labeled with said universal reagent;
(c) contacting said plurality of complexes with excess reporter reagent, the reporter reagent having an affinity for at least a portion of said universal reagent and further comprised of a detectable label, under conditions effective to provide a plurality of complexes, each complex bearing a detectable label or cleavable group;
(d) detecting through said detectable label the existence of a complex and determining through said distinguishable secondary reagent the nature of said given analyte found therein, thereby revealing the identities of the plurality of different analytes present in the fluid sample; and
(e) compiling analyte data generated from step (d) in a database.

37. The method of claim 36 in which said database is in a computer readable format.

38. A method of generating information useful in the creation of a diagnostic assay for arriving at a likelihood that a subject is suffering from or will later develop one or more diseases comprising:
(a) contacting a fluid sample obtained from a subject suffering from or who later develops one or more diseases, which fluid sample is suspected of harboring a plurality of different analytes, with a universal reagent capable of binding to all or substantially all of said plurality of different analytes, under conditions effective to label all or substantially all of said plurality of different analytes with said universal reagent;
(b) contacting said plurality of different analytes labeled with said universal reagent with a collection of distinguishable subpopulations of secondary reagent, each secondary reagent of a given distinguishable subpopulation being designed to interact selectively with a given analyte of said plurality of different analytes, under conditions effective to provide a plurality of complexes, each complex comprised of said distinguishable secondary reagent and said given analyte labeled with said universal reagent;
(c) detecting through said detectable label the existence of a complex and determining through said distinguishable secondary reagent the nature of said given analyte found therein, thereby revealing the identities of the plurality of different analytes present in the fluid sample;
(d) repeating steps (a) to (c) using fluid samples from a sufficient number of subjects suffering from or who later develop one or more diseases to provide a database of accumulated analyte data comprising a compilation of analyte data obtained from a statistically significant number of subjects suffering from or who later develop one or more diseases;
(e) extracting information from said database, which information is useful in the creation of a diagnostic assay for arriving at a likelihood that a subject is suffering from or will later develop one or more diseases.

39. The method of claim 37 which further comprises selecting a set of given analytes the presence or absence of which appears to correlate with a likelihood that a subject is suffering from or will later develop one or more diseases.

40. The method of claim 37 in which all the subjects chosen for inclusion in the database suffer from or will later develop the same or similar such one or more diseases.
